(19) United States
(12) Patent Application Publication
(10) Pub. No.: US 2023/0077029 A1
(43) Pub. Date: Mar. 9, 2023

(54) Systems and Methods to Optimize the Bone Implant Interface

(71) Applicant: Mayo Foundation for Medical Education and Research, Rochester, MN (US)

(72) Inventor: John W. Sperling, Rochester, MN (US)

(21) Appl. No.: 17/799,147

(22) PCT Filed: Feb. 12, 2021
(86) PCT No.: PCT/US2021/017817

§ 371 Date: Aug. 11, 2022

Related U.S. Application Data
(60) Provisional application No. 62/975,458, filed on Feb. 12, 2020.

(51) Int. Cl. A61F 2/28; A61F 2/30

(52) U.S. Cl. CPC A61F 2/28; A61F 2/30942; A61F 2002/2853; A61F 2002/30069; A61F 2002/30952

(57) ABSTRACT

Systems and methods are provided for implant design and manufacturing to optimize the bone-implant interface. The implant design and methodology may include accounting for the anatomy of a bone of a subject to address optimize the bone-implant interface considerations for the subject. Implants or components may be asymmetrically designed to better match the associated anatomy as well as optimize the bone-implant interface, such as by quantifying bone density and matching material properties of the implant or coatings of the implant. Information derived from the methodology can be used to guide the design of the implant resulting in an asymmetric design that optimizes the bone-implant interface.

### CROSS-REFERENCE TO RELATED APPLICATIONS

This application claims the benefit of U.S. Provisional Patent Application Ser. No. 62/975,458 filed on Feb. 12, 2020 and entitled “Systems and Methods to Reduce Stress Shielding in Implants,” which is incorporated herein by reference as if set forth in its entirety for all purposes.

### STATEMENT REGARDING FEDERALLY SPONSORED RESEARCH

N/A

### BACKGROUND

There has been a dramatic increase in the use of orthopedic and dental implants worldwide. Studies across anatomic areas have demonstrated that one of the central problems is component loosening.

Implants may loosen due to a lack of strong initial fixation at the bone-implant interface as well as due to non-uniform stress distribution. One manifestation of non-uniform stress distribution is stress shielding. Stress shielding refers to the reduction in bone density (osteopenia) as a result of removal of typical stress from bone by an implant (for instance, the stem of a joint prosthesis). This is because by Wolff's law, bone in a healthy person or animal will remodel in response to the loads it is placed under. After surgery, the bone shares its load with the implant. However, the modulus of elasticity varies between the metal and bone, resulting in changes of how the forces are distributed. Therefore, as the loading on a bone decreases, the bone will become less dense and weaker with resultant bone resorption. The magnitude of stress shielding and the specific locations of bone loss are based on the differences between the properties of the implant and the properties of the underlying bone.

There is extensive literature reporting that stress shielding can result in severe bone resorption across anatomic sites. For example, in shoulder replacement, short stem uncemented humeral components have been reported to have rates of bone resorption of greater than 40 to 70% in short term follow-up. In longer term studies, at a mean of 8 years, one reference reported stress shielding in 47% uncemented stems with partial or complete greater tuberosity resorption in 100% uncemented stems.

Stress shielding also occurs when plates or intramedullary nails are used to repair fractures. While the rigid nature of plates and nails helps to stabilize the fracture and facilitates early mobility, the increased stiffness of the plate or intramedullary nail results in bone loss due to decreased loading of the bone. It has been shown that bone remodeling is extremely sensitive to even small changes in cyclic bone stresses. Changes in cyclic bone stresses of even less than 1% of the ultimate strength can result in measurable changes in bone remodeling after only a few months.

Orthopedic and dental implants function as rigid osseous anchors within bone. The mechanical behavior of implant materials, surface coatings, and shape result in adaptive bone remodeling. The mechanical mismatch between host bone and metallic implants has been a long-lasting concern. For example, the elastic modulus of bone is 10-30 GPa. For two of the most commonly used implant materials, the elastic modulus is approximately 100 GPa for pure titanium and 230 GPa for cobalt-chrome. In this environment, contacted bone is often inappropriately stress shielded, and hence, implants lose supportive bone at the implant-bone region over time.

The current practice in orthopedics has been the use of implants that have been designed without regard to the variability of internal bone architecture. For example, the design and manufacturing of stems for joint arthroplasty has been traditionally driven by ease of and cost of manufacturing. Therefore, humeral stems and stemless implants have been designed to be symmetric and can be used in right or left sides. This minimizes inventory as well as the cost of manufacturing. This long held practice has been forcing the anatomy to fit the implant rather than making the implant match the anatomy and underlying bone architecture.

These concerning findings highlight the need for technology and strategies to address the causes of implant loosening with technology and strategies to optimize the bone-implant interface. These strategies can include improved immediate implant fixation as well as improved stress distribution at the bone-implant interface.

### SUMMARY OF THE DISCLOSURE

The present disclosure addresses the aforementioned drawbacks by providing systems and methods for implant design and manufacturing to optimize the bone-implant interface. These strategies can improve the immediate fixation as well as the stress distribution at the bone-implant interface. The implant design and methodology may include taking into account the anatomy of a bone of a subject to optimize the bone-implant interface considerations for the subject. The implant design may be asymmetric, and/or may include asymmetric bone-growth promoting coatings applied at locations determined to optimize the bone-implant interface.

In one configuration, a method is provided for manufacturing an orthopedic implant for repairing a part of a bone in a subject. The method includes forming the implant to include at least one material property determined by: i) obtaining an image of the bone from at least one viewing plane; ii) orienting on the image a cross section indicating a maximum width of a feature of the bone from a first border of the bone to an opposite second border of the bone; and iii) determining the at least one material property to optimize the bone-implant interface using the maximum width of the feature of the bone. In one configuration, determining the at least one material property may include to reduce stress shielding using the maximum width of the feature of the bone.

In some configurations, the material property includes at least one of elasticity, surface coating treatment, porosity, thickness, or shape. The material property may create an asymmetric implant. The material property may include the surface coating treatment of the implant, which may correspond to an anatomic location determined by a location of the cross section of the bone. The surface coating treatment may be asymmetric on the surface of the implant.

In some configurations, the method includes determining a quality of the bone using the maximum width of the feature of the bone. Determining the quality of the bone may include determining a score for the quality of the bone in an anatomic location.

In some configurations, a plurality of cross sections are oriented on the image along a longitudinal axis of the bone. Bone quality may be determined for a plurality of features along the longitudinal axis of the bone using the maximum width of each of the plurality of features. In some configurations, the bone is a humerus and a joint that includes the bone is a shoulder.

In one configuration, a device is provided for repairing a part of a bone in a subject. The device includes a first section having a first material property and a second section having a second material property. The first section is connected to the second section forming an asymmetric implant, where the first section and the second section are configured to optimize the bone-implant interface. In one configuration, the first section and the second section are configured to reduce stress shielding.

In some configurations, the first material property includes at least one of elasticity, surface coating treatment, porosity, thickness, or shape. The second material property may include at least one of elasticity, surface coating treatment, porosity, thickness, or shape. In some configurations, at least one of the first material property or the second material property includes a surface coating treatment of the device, that corresponds to a location of uneven stress distribution. The surface coating treatment may be asymmetric on the surface of the device. The location of uneven stress distribution of the bone may correspond to a location of reduced bone thickness. The bone may be a humerus and a joint that includes the bone may be a shoulder.

In one configuration, a device is provided for repairing a part of a bone in a subject. The device includes a first section having a first material property and a second section having a second material property. The first section is connected to the second section. The first material property or the second material property includes a surface coating treatment forming an asymmetric implant and is configured to optimize the bone-implant interface. In one configuration, the first material property or the second material property includes a surface coating treatment forming an asymmetric implant and is configured to reduce stress shielding.

In some configurations of the device, the first material property is a surface coating treatment and the second material property includes at least one of elasticity, porosity, thickness, or shape. The second material property may include a surface coating treatment and the first material property may include at least one of elasticity, porosity, thickness, or shape. The surface coating treatment may correspond to a location of uneven stress distribution of the bone. The location of the uneven stress distribution of the bone may correspond to a location of reduced bone thickness. In some configurations of the device, the bone is a humerus, and a joint that includes the bone is a shoulder.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a flowchart of non-limiting example steps for configuring a material property of an implant to optimize the bone-implant interface.

FIG. 2 shows a traced computed tomography (CT) two-dimensional (2D) CT slice in a coronal viewing plane of the humerus with measurement lines shown in dashed lines.

FIG. 2A shows a traced computed tomography (CT) two-dimensional (2D) CT slice in a coronal viewing plane of the humerus.

FIG. 2B shows a traced computed tomography (CT) two-dimensional (2D) CT slice in a sagittal viewing plane of the humerus with measurement lines shown in dashed lines.

FIG. 2C shows a traced computed tomography (CT) two-dimensional (2D) CT slice in a sagittal viewing plane of the humerus.

FIG. 2D shows an axial cross section of a humerus with a depiction of the cross section's location on an image of a humerus bone in FIG. 2E with measurement lines shown.

FIG. 2E is an image of a humerus bone with the location of the cross section from FIG. 2D shown.

FIG. 3A is a graphical representation of non-limiting example cancellous anterior to posterior width measurements acquired for humeral bones.

FIG. 3B is a graphical representation of non-limiting example cancellous medial to lateral width measurements acquired for humeral bones.

FIG. 3C is a graphical representation of non-limiting example cortical anterior to posterior width measurements acquired for humeral bones.

FIG. 3D is a graphical representation of non-limiting example cortical medial to lateral width measurements acquired for humeral bones.

FIG. 4A is a graphical representation of non-limiting example cancellous anterior to posterior width measurements acquired for tibia bones.

FIG. 4B is a graphical representation of non-limiting example cancellous medial to lateral width measurements acquired for tibia bones.

FIG. 4C is a graphical representation of non-limiting example cortical anterior to posterior width measurements acquired for tibia bones.

FIG. 4D is a graphical representation of non-limiting example cortical medial to lateral width measurements acquired for tibia bones.

FIG. 5A is a graphical representation of non-limiting example cancellous anterior to posterior width measurements acquired for femur bones.

FIG. 5B is a graphical representation of non-limiting example cancellous medial to lateral width measurements acquired for femur bones.

FIG. 5C is a graphical representation of non-limiting example cortical anterior to posterior width measurements acquired for femur bones.

FIG. 5D is a graphical representation of non-limiting example cortical medial to lateral width measurements acquired for femur bones.

FIG. 6A is a graphical representation of non-limiting example cancellous anterior to posterior width measurements acquired for fibula bones.

FIG. 6B is a graphical representation of non-limiting example cancellous medial to lateral width measurements acquired for fibula bones.

FIG. 6C is a graphical representation of non-limiting example cortical anterior to posterior width measurements acquired for fibula bones.

FIG. 6D is a graphical representation of non-limiting example cortical medial to lateral width measurements acquired for fibula bones.

FIG. 7A is a non-limiting example cross section of a symmetrical implant with a coating applied in accordance with the present disclosure.

FIG. 7B is a non-limiting example cross section of an asymmetrical implant with a coating applied in accordance with the present disclosure.

FIG. 8A is a non-limiting example cross section of a symmetrical stem with a coating applied in accordance with the present disclosure.

FIG. 8B is a non-limiting example cross section of an asymmetrical stem with a coating applied in accordance with the present disclosure.

### DETAILED DESCRIPTION

Systems and methods are provided for implant design and manufacturing to optimize the bone-implant interface. The implant design and methodology may include taking into account the anatomy of a bone of a subject to address bone-implant interface considerations for the subject. Implants or components can be asymmetrically designed to better match the associated anatomy as well as to optimize the bone-implant interface, such as by quantifying bone density and matching material properties of the implant. Information derived from the methodology can be used to guide the design of the implant resulting in an asymmetric design that optimizes the bone-implant interface. In some configurations, a method for implant design to optimize the bone-implant interface includes an asymmetrical shape of the implant; an asymmetric coating applied to the implant, an asymmetric type of coating applied to the implant; an asymmetric modulus of elasticity of the implant material, and the like. Implants may be configured for repairing a part of a bone in a subject, such as by repairing a fracture, or through arthroplasty, and the like.

Previously, the shape, texturing, as well as material properties for implants like the humerus have been symmetric without regard for the relative thickness of the cortical and cancellous bone. The central flaw and deficiency of symmetric implants is that the implants do not match the anatomy. Anatomy, however, is not symmetric. There are dramatic differences in the thickness of cortical and cancellous bone based on the specific location within the bone.

In some configurations, cortical and cancellous thickness can be measured in cross-sections at any interval down a bone. Measurements of thickness can also be performed in any angular direction from the center of the bone. With such measurements, a true three dimensional thickness of cortical and cancellous bone in a 3-dimensional quantifiable manner may be possible for a bone. The shape and material properties of the implant may then be tailored to match the three dimensional architecture of the bone.

In some configurations, the current methodology facilitates the ability to quantify the thickness of bone at specific locations within the bone. The quantified thickness may be of cancellous or cortical bone. In a non-limiting example, images of a bone may be divided into specific levels. At each level, specific zones may be defined. In some configurations, the quality of the bone in each zone may be quantified. This quantification may be performed using a medical imaging system, such as radiography, CT scan, densitometry, dual energy x-ray absorptiometry (DEXA), and the like. In each zone, a user or an automated system may score the material property of the bone by using the thickness of the specific component of the bone (cortical and/or cancellous) and/or bone quality. This score may then guide the material properties in a corresponding specific region of the implant. The methodology allows the implant to better match the underlying bone with more uniform stress distribution by customizing the implant or a coating of the implant at each corresponding location of the bone. This information can be used to customize the implant for an individual patient. This information could also be generated on a population of patients and implants can then be tailored to specific patient populations.

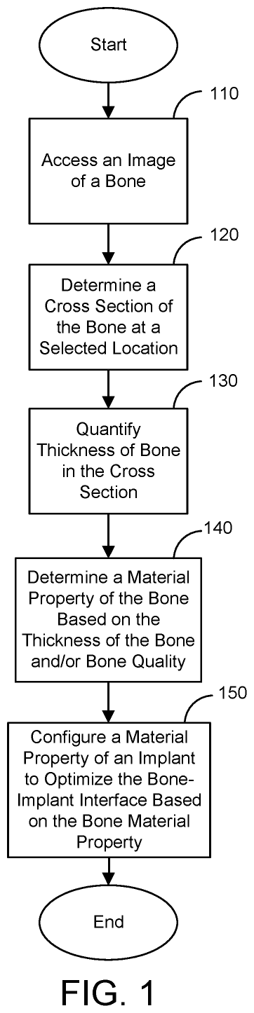

Referring to FIG. 1, non-limiting example steps are shown for a method for adapting an implant to optimize the bone-implant interface in a bone of a subject. An image of a bone may be obtained from an image archive, acquired using a medical imaging system, or otherwise accessed at step 110. Cross sections of the bone may be determined at step 120 for selected locations along the bone. The thickness of a bone, such as cortical bone or cancellous bone, may be determined for the cross sections at step 130. A material property for the bone, such as a quantity, strength, quality, and the like, may be determined based on the thickness of the bone and/or bone quality at step 140. In a non-limiting example, the material property is bone quality, and a score may be generated as an indicator of the bone quality, with higher scores indicating higher bone quality. Lower bone quality may be reflected by a lower score. In another non-limiting example, a score that takes into consideration both the thickness of the bone and the quality of the bone can be generated. In a non-limiting example, a scoring range can then be obtained with 1 being low and 10 being high. A material property of an implant, such as elasticity, surface coating treatment, porosity and the like, may be configured in step 150 to optimize the bone-implant interface based upon the determined bone material property from step 140, or the determined score.

In one configuration, a score that quantifies the thickness of the bone and/or the quality of the bone may be determined based upon a location in a cross section of the bone. Different radial locations in the same cross section of the bone may have different scores and therefore would have differing amounts of intervention to optimize the bone-implant interface, such as bone in-growth coatings to an implant and the like. In a non-limiting example, a score for the thickness of a cortical and/or cancellous bone at a radial location of a cross section of a bone may be determined by using the centerline of the bone as the origin of the radial coordinates. The bone may vary in thickness or bone quality at different radial locations in the cross section and thereby different scores may be determined for different radial locations of the cross section of bone.

In another configuration, a score may be determined qualitatively using a clinical feedback that assesses the thickness of the bone and/or the quality of the bone based upon a location in a cross section of the bone and clinical knowledge of the stress distribution effects to be expected. Different radial locations in the same cross section of the bone may have different scores and therefore would have differing amounts of intervention to optimize the bone-implant interface, such as through application of bone in-growth coatings to an implant and the like.

Significant variability exists in thickness of cortical and cancellous bone based on the specific location within the bone. This understanding may be applied to the manufacture of implants in accordance with the present disclosure. An implant may be configured to better match the variability that exists in the thickness of the cortical and cancellous bone, as well as quality of the bone, facilitating more even stress transfer and optimizing the bone-implant interface.

Optimization may also include increasing the immediate torque-out strength and optimal loading of an implant, as described in U.S. Patent Application Publication No. 2019/0105169, which is incorporated by reference herein in its entirety for all purposes. A testing protocol for quantifying resistance to torque-out failure can be found in U.S. 2019/0105169. Increasing the immediate torque-out strength may include increasing the torque-out strength of the implant immediately following implantation and prior to any significant resulting bone in-growth. The torque-out strength may be increased above a threshold value, where the threshold value is the minimum value for a symmetrical, non-optimized implant to remain fixed upon implantation. After implantation, optimization may also include optimizing longer term and more even stress distribution between the bone and the implant.

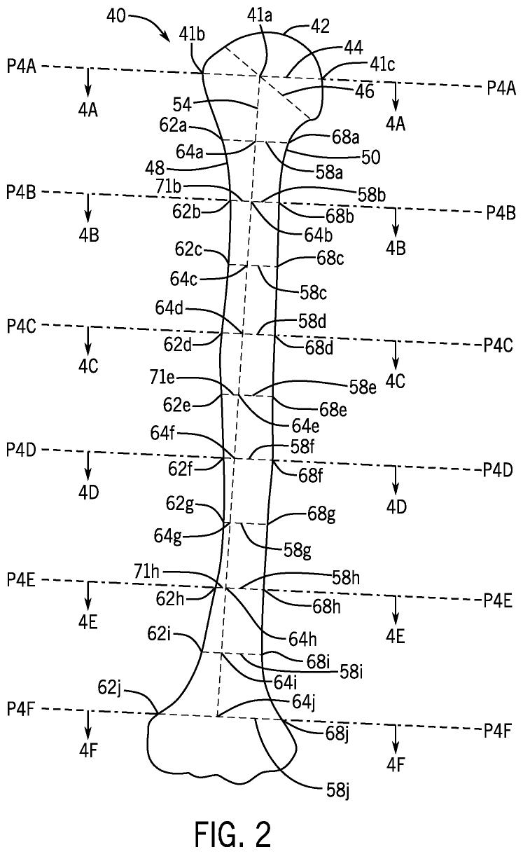

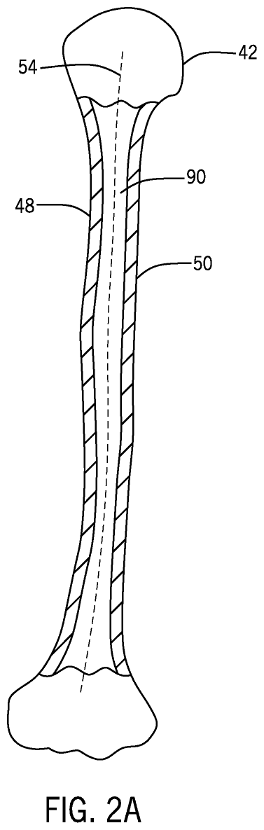

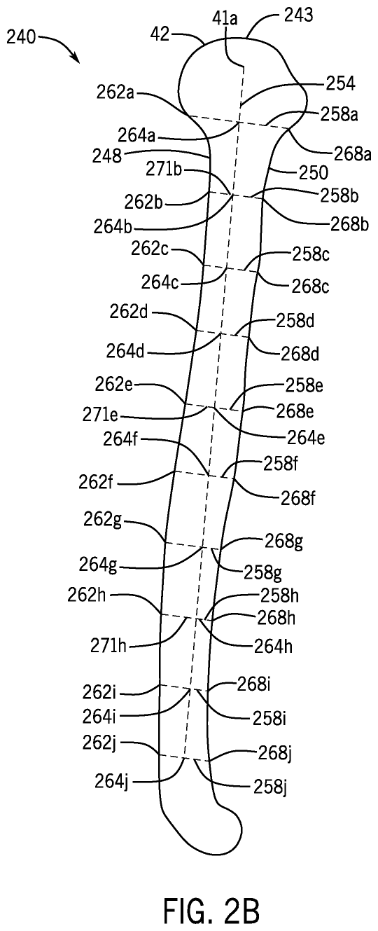

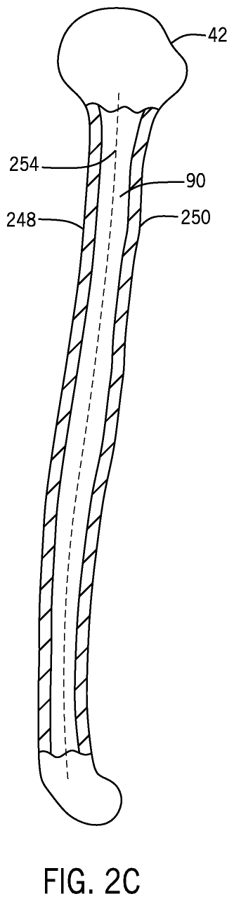

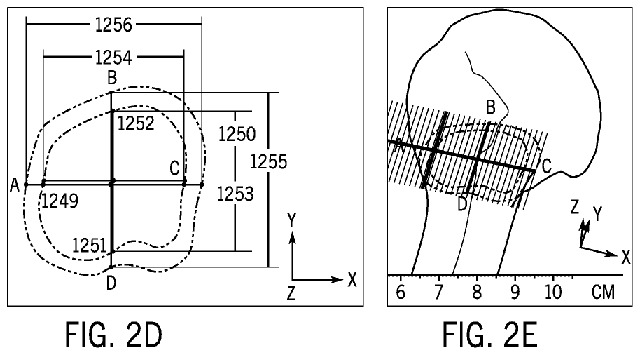

Looking at FIG. 2, the anatomic shape of a bone and thereby the asymmetric configuration for an implant can be determined by a number of steps. An image 40 of a bone 42 of a subject can be obtained, where in some embodiments the image 40 can be a CT image, in other embodiments the image can be an X-ray image, an ultrasonic image, a magnetic resonance image (MRI), a positron emission tomography (PET) image, or the like. The bone can be a femur, tibia, fibula, and the like. In other embodiments, the bone can be a radius, an ulna, or any other bone. A bone cut line 46 can be oriented on the image 40 that can extend from a first border 48 of the bone 42 to an opposite second border 50 of the bone 42. In some embodiments, the bone cut line 46 can be oriented angularly across a region of a head of the bone of a subject. A longitudinal bone axis 54 can be oriented on the image 40. The longitudinal bone axis 54 may extend longitudinally from a proximal aspect of the bone. In another embodiment, the longitudinal bone axis 54 may extend longitudinally from an intersection 41A of a proximal aspect line, such as proximal bone head line 44 with the bone cut line 46, where the proximal bone head line 44 is oriented on the image 40 by extending perpendicularly from a first intersection point 41B on the first border 48 of the bone 42 at the most proximal and lateral aspect of a greater tuberosity through a second intersection point 41A where the proximal bone head line 44 intersects the bone cut line 46, and further extends to a third intersection point 41C on the second border 50 of the bone 42. In some embodiments, the bone 42 can be the femur, or the tibia, or the fibula.

Referring to FIG. 2A, one embodiment is shown where the longitudinal bone axis 54 can follow the centerline of bone 42, defined as being a constant equal distance between the first border 48 and the second border 50. When bone axis 54 is the centerline of the bone 42, the nonlinear shape of the axis line 54 defines the radius of curvature for the bone 42, which can be assessed at various points along the length of the bone axis line 54. The nonlinear shape of the axis line 54 can provide a number of different radii of curvature. When bone axis 54 is the centerline, the intersection of axis line 54 with proximal bone head line 44 may determine intersection point 41A. Any number of changes in radius of curvature can be provided such that the axis line 54 is a constant equal distance between the first border 48 and the second border 50 within the intramedullary canal 90 of the cancellous bone.

Referring again to FIG. 2, in another embodiment the longitudinal bone axis 54 does not follow the centerline, but may be linear and can extend from the bone cut line 46, or may extend linearly from intersection point 41A that was established from the intersection of a centerline with proximal greater tuberosity line 44, along a length of the bone between the first border 48 and second border 50. A plurality of lateral lines 58a, 58b, 58c, 58d, 58e, 58f, 58g, 58h, 58i, 58j can be oriented on the image 40 at different distances from the intersection point 41a, or from a proximal aspect line, such as proximal greater tuberosity line 44, or from the bone cut line 46. Each of the plurality of lateral lines 58a to 58j can extend perpendicularly from one of a plurality of first intersection points 62a, 62b, 62c, 62d, 62e, 62f, 62g, 62h, 62i, 62j on the first border 48 of the bone 42 to one of a plurality of second intersection points 64a, 64b, 64c, 64d, 64e, 64f, 64g, 64h, 64i, 64j intersecting the longitudinal bone axis 54 at one of a plurality of second intersection points 64a to 64j. Each of the plurality of lateral lines 58a to 58j can further extend perpendicularly from one of a plurality of second intersection points 64a to 64j on the longitudinal bone axis 54 to one of a plurality of third intersection points 68a, 68b, 68c, 68d, 68e, 68f, 68g, 68h, 68i, 68j on the second border 50 of the bone 42. The anatomic shape of the bone 42 can be extrapolated based on determining the first intersection point 41B of the proximal greater tuberosity line 44 along with the plurality of first intersection points 62a to 62j, and measuring the distances to the corresponding second intersection points, which for intersection point 41B would be intersection point 41A of the proximal greater tuberosity line 44, and subsequently the plurality of second intersection points 64a to 64j from the first intersection points 62a to 62j. Specifically, the anatomic shape of the first border 48 of the bone 42 can be extrapolated from the first intersection point 41B of the proximal greater tuberosity line 44 and with the plurality of first intersection points 62a to 62j with the plurality of second intersection points 41a and 64a to 64j. The anatomic shape of the second border 50 of the bone 42 can be extrapolated in a similar manner as above by using the third intersection point 41C of the proximal greater tuberosity line 44 with the plurality of third intersection points 68a to 68j and measuring the distances to the corresponding second intersection points 41a and 64a to 64j.

In some embodiments, the plurality of lateral lines 58a to 58j can be placed at equidistant intervals distally from intersection point 41a, or from a proximal aspect line, such as proximal greater tuberosity line 44, or from the bone cut line 46. In some embodiments, the equidistant interval can be in a range from 0.1 to 50 millimeters. In a non-limiting embodiment, the equidistant interval can be 25 millimeters. As such, example measurements can be made at 25 millimeters, 50 millimeters, 75 millimeters, 100 millimeters, 125 millimeters, 150 millimeters, 175 millimeters, and 200 or more millimeters distal to the intersection point 41a, or from a proximal aspect line, such as proximal greater tuberosity line 44, or from the bone cut line 46. One can add more lines to provide for determining the contour of the bone with higher resolution.

In a non-limiting example embodiment, a first reference distance can be measured for a first line 71b extending perpendicularly from a first point 62b of the plurality of first intersection points 62a to 62h to a first point 64b of the plurality of second intersection points 64a to 64h. A second reference distance can be measured of a second line 71e extending perpendicularly from a second point 62e of the plurality of first intersection points 62a to 62h to a second point 64e of the plurality of second intersection points 64a to 64h. A third reference distance can be measured of a third line 71h extending perpendicularly from a third point 62h of the plurality of first intersection points 62a to 62h to a third point 64h of the plurality of second intersection points 64a to 64h.

The anatomic shape of the first border 48 can be extrapolated based on the first reference distance of the first line 71b, the second reference distance of the second line 71e, and the third reference distance of the third line 71h. A first curvature of the anatomic shape can be extrapolated between the first point 62b of the plurality of first intersection points 62a to 62h and the second point 62e of the plurality of first intersection points 62a to 62h based on the first reference distance and the second reference distance. A second curvature of the anatomic shape can be extrapolated between the second point 62e of the plurality of first intersection points 62a to 62h and the third point 62h of the plurality of first intersection points 62a to 62h based on the second reference distance and the third reference distance.

In another version of the method of the disclosure, the anatomic shape of the first border 48 and the second border 50 together can be extrapolated based on a fourth reference distance of the lateral line 58b, a fifth reference distance of the lateral line 58e, and a sixth reference distance of the lateral line 58h. A first curvature of the anatomic shape can be extrapolated between the first point 62b of the plurality of first intersection points 62a to 62j and the second point 62e of the plurality of first intersection points 62a to 62j based on the fourth reference distance and the fifth reference distance. A second curvature of the anatomic shape can be extrapolated between the second point 62e of the plurality of first intersection points 62a to 62j and the third point 62h of the plurality of first intersection points 62a to 62j based on the fifth reference distance and the sixth reference distance.

Looking at FIG. 2B, the anatomic shape can be determined by a number of steps. An image 240 of a bone 42 of a subject can be obtained in a sagittal viewing plane; in some embodiments the image 240 can be a CT image; in other embodiments the image can be an X-ray image, an ultrasonic image, a magnetic resonance image (MRI), a positron emission tomography (PET) image, or the like. The bone 42 can be a humerus. In other embodiments the bone can be a radius, an ulna, a femur, a tibia, or any other bone.

A longitudinal bone axis 254 can be oriented on the image 240. The longitudinal bone axis 254 may extend from intersection point 41a from FIG. 2 along a length of the bone 42 between a first border 248 and a second border 250. FIG. 2C shows one embodiment where the longitudinal bone axis 254 can follow the centerline of bone 42, defined as being a constant equal distance between the cortical bone first border 248 and the second border 250. Alternatively, longitudinal bone axis 254 may be the centerline of bone 42, being defined as a constant equal distance between the borders of the cancellous bone, which would take into account any differences with cortical bone thickness. When bone axis 254 is the centerline of the bone 42, the nonlinear shape of the axis line 254 defines the radius of curvature for the bone 42, which can be assessed at various points along the length of the bone axis line 254. The nonlinear shape of the axis line 254 can provide a number of different radii of curvature. In one non-limiting example, a first radius of curvature can transition to a second radius of curvature, and the second radius of curvature can transition to a third radius of curvature. The first radius of curvature and the third radius of curvature can be concave, while the second radius of curvature can be convex. Each radius of curvature can feature a different radius. Any number of changes in radius of curvature can be provided such that the axis line 254 is a constant equal distance between the first border 248 and the second border 250 within the intramedullary canal 90 of the cancellous bone. In one embodiment, the deviation from the straight longitudinal bone axis 254 (FIG. 2B) from the centerline following bone axis 254 (FIG. 2C) may be determined in order to indicate where over the length of the bone the area or areas of greatest bending or deflection take place. This may be used when designing plates, intramedullary nails, stems, or other implants for bends that may be needed to conform to the anatomy, and in guiding the locations for where uneven stress distribution may be more likely to occur.

A plurality of lateral lines 258a, 258b, 258c, 258d, 258e, 258f, 258g, 258h, 258i, 258j can be oriented on the image 240 at different distances from the proximal end 243, or from point 41a. Each of the plurality of lateral lines 258a to 258j can extend perpendicularly from one of a plurality of first intersection points 262a, 262b, 262c, 262d, 262e, 262f, 262g, 262h, 262i, 262j on the first border 248 of the bone 42 to one of a plurality of second intersection points 264a, 264b, 264c, 264d, 264e, 264f, 264g, 264h, 264i, 264j intersecting the longitudinal bone axis 254 at one of a plurality of second intersection points 264a to 264j. Each of the plurality of lateral lines 258a to 258j can further extend perpendicularly from one of a plurality of second intersection points 264a to 264j on the longitudinal bone axis 254 to one of a plurality of third intersection points 268a, 268b, 268c, 268d, 268e, 268f, 268g, 268h, 268i, 268j on the second border 250 of the bone 42. The anatomic shape of the bone 42 can be extrapolated based on the plurality of first intersection points 262a to 262j, and the plurality of second intersection points 264a to 264j. Specifically, the anatomic shape of the first border 248 of the bone 42 can be extrapolated from the plurality of first intersection points 262a to 262j and the plurality of second intersection points 264a to 264j. The anatomic shape of the second border 250 of the bone 42 can be extrapolated from the plurality of second intersection points 264a to 264j and the plurality of third intersection points 268a to 268j.

In some embodiments, the plurality of lateral lines 258a to 258j can be placed at equidistant intervals distally from the proximal end 243. In some embodiments, the equidistant interval can be in a range from 0.1 to 50 millimeters. In a non-limiting embodiment, the equidistant interval can be 25 millimeters. As such, example measurements can be made at 25 millimeters, 50 millimeters, 75 millimeters, 100 millimeters, 125 millimeters, 150 millimeters, 175 millimeters, and 200 or more millimeters distal to the proximal end 243. One can add more lines to provide for determining the contour of the bone with higher resolution.

In a non-limiting example embodiment, a first reference distance can be measured for a first line 271b extending perpendicularly from a first point 262b of the plurality of first intersection points 262a to 262h to a first point 264b of the plurality of second intersection points 264a to 264h. A second reference distance can be measured of a second line 271e extending perpendicularly from a second point 262e of the plurality of first intersection points 262a to 262h to a second point 264e of the plurality of second intersection points 264a to 264h. A third reference distance can be measured of a third line 271h extending perpendicularly from a third point 262h of the plurality of first intersection points 262a to 262h to a third point 264h of the plurality of second intersection points 264a to 264h.

The anatomic shape of the first border 248 can be extrapolated based on the first reference distance of the first line 271b, the second reference distance of the second line 271e, and the third reference distance of the third line 271h. A first curvature of the anatomic shape can be extrapolated between the first point 262b of the plurality of first intersection points 262a to 262h and the second point 262e of the plurality of first intersection points 262a to 262h based on the first reference distance and the second reference distance. A second curvature of the anatomic shape can be extrapolated between the second point 262e of the plurality of first intersection points 262a to 262h and the third point 262h of the plurality of first intersection points 262a to 262h based on the second reference distance and the third reference distance.

In some configurations, the anatomic shape of the first border 248 and the second border 250 together can be extrapolated based on a fourth reference distance of the lateral line 258b, a fifth reference distance of the lateral line 258e, and a sixth reference distance of the lateral line 258h. A first curvature of the anatomic shape can be extrapolated between the first point 262b of the plurality of first intersection points 262a to 262j and the second point 262e of the plurality of first intersection points 262a to 262j based on the fourth reference distance and the fifth reference distance. A second curvature of the anatomic shape can be extrapolated between the second point 262e of the plurality of first intersection points 262a to 262j and the third point 262h of the plurality of first intersection points 262a to 262j based on the fifth reference distance and the sixth reference distance.

Referring to FIG. 2D, the plurality of lateral lines 58a, 58b, 58c, 58d, 58e, 58f, 58g, 58h, 58i, 58j from FIG. 2 or FIG. 2A may include being placed in multiple planes, such as A-C and D-B in FIG. 2D. The plurality of lateral lines may also include measurements of the thickness of the cortical and cancellous bone material. In the example provided, thicknesses for the cortical and cancellous bone is obtained in a 2D cross section of the humerus, such as the cross section depicted in FIG. 2E. Cortical lateral thickness 1249, cortical medial thickness 1250, cortical anterior thickness 1251, and cortical posterior thickness 1252 may be determined. Cancellous anterior to posterior distance 1253, cancellous medial to lateral distance 1254, cortical anterior to posterior distance 1255, and cortical medial to lateral distance 1256 may also be determined. In some embodiments, any orientation for the planes may be used, such as a partially rotated anterior to a partially rotated posterior view, which may enable for fully 3D thickness measurements of the bone. In one embodiment, these measurements may be obtained in an automated fashion, where a medical image is provided to a computer system that automatically segments the bone, identifies the relevant anatomical landmarks, such as the humeral head and the olecranon fossa, and performs the desired measurements.

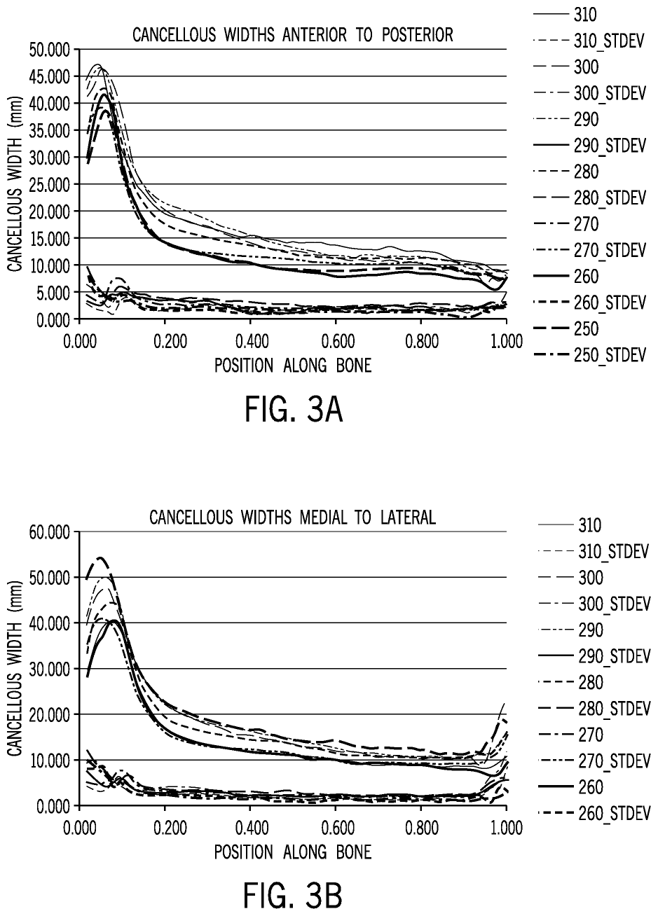

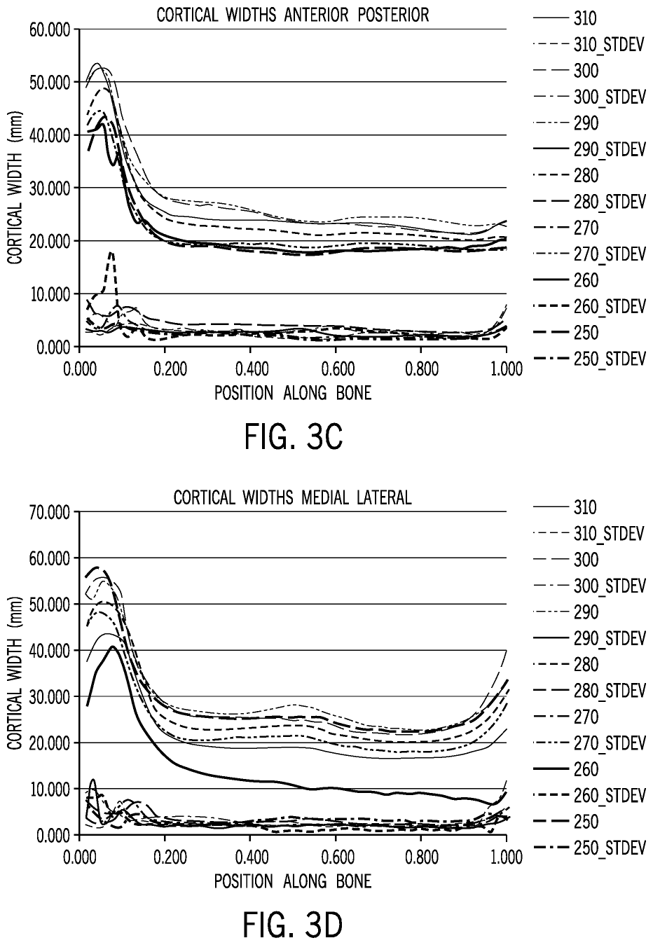

Referring to FIGS. 3A, 3B, 3C, and 3D graphs of non-limiting example humeral bone thickness data is shown. Bone thickness data may be used to determine an implant design by indicating areas that may result in future uneven stress distribution. In some configurations bone thickness is a surrogate metric for bone quality, and so thickness data such as presented in the current figures may also be used to determine regions of a bone that may experience uneven stress distribution. Regions of the bone that might experience uneven stress distribution may benefit from a differential thickness of bone-growth material being present on the surface of the implant at that corresponding location compared to other bone regions. An implant may also be positioned on/in the bone in order to maximize contact with thicker bone.

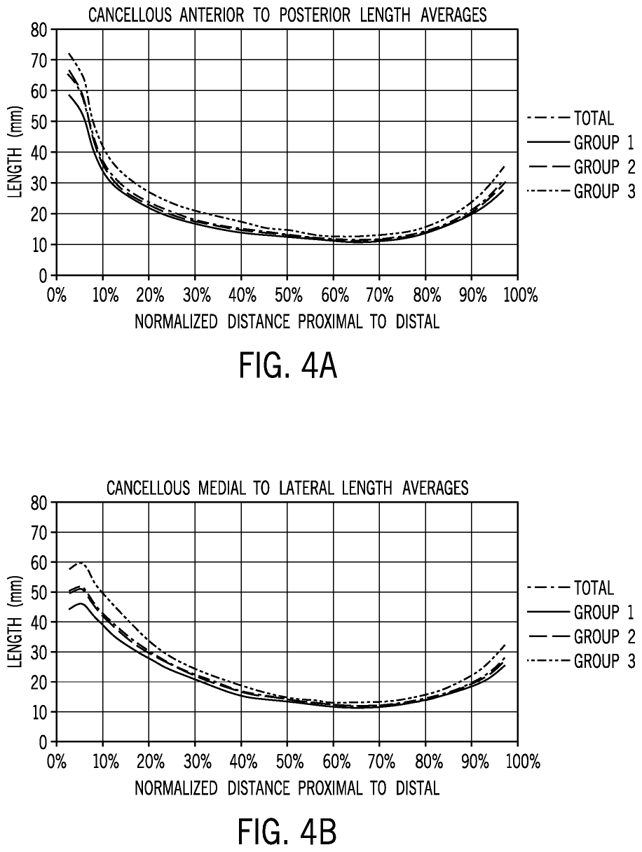

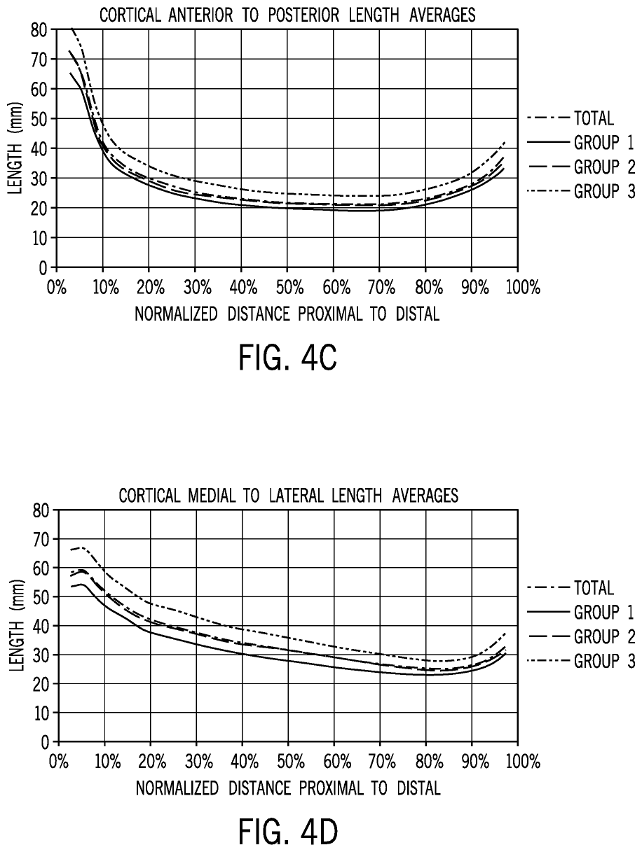

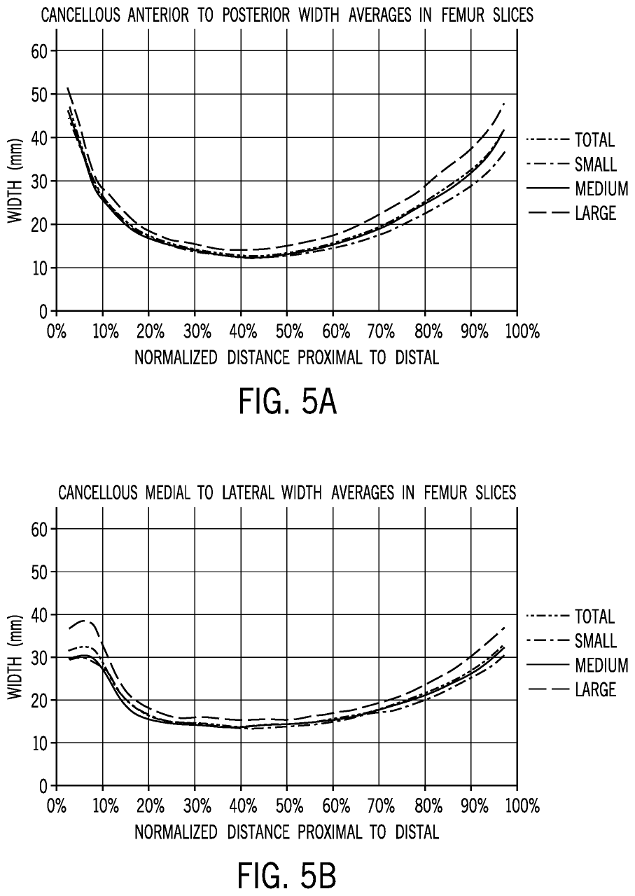

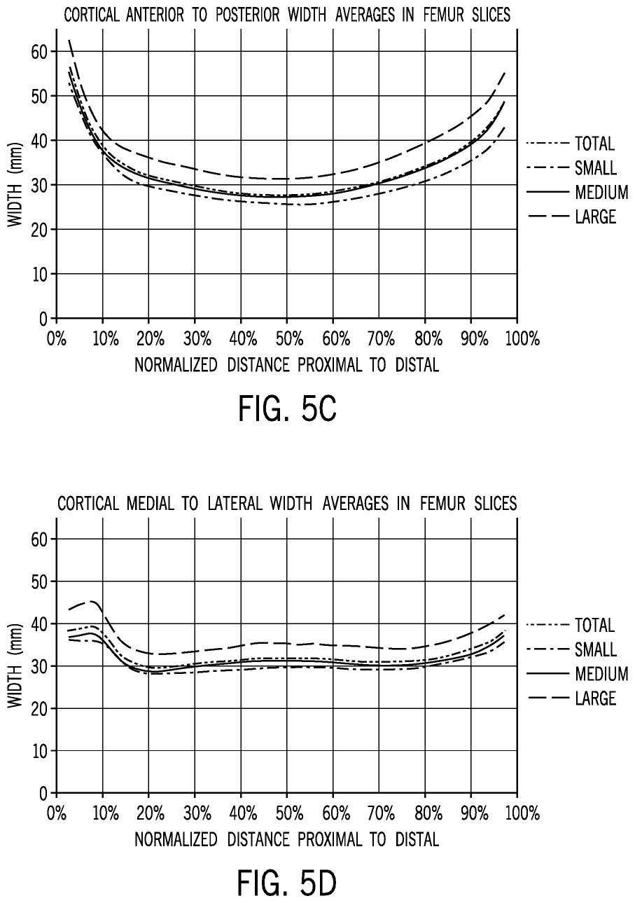

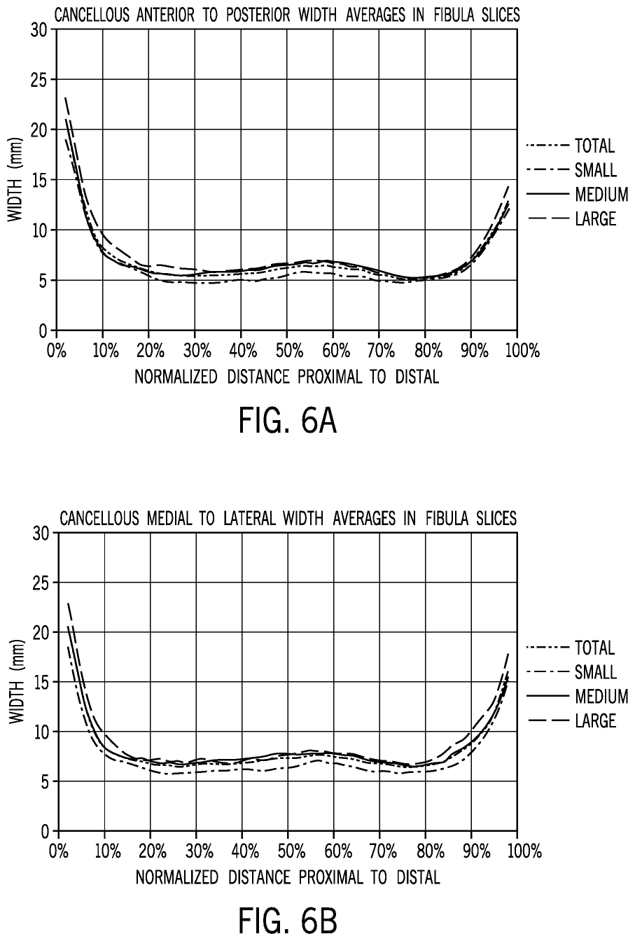

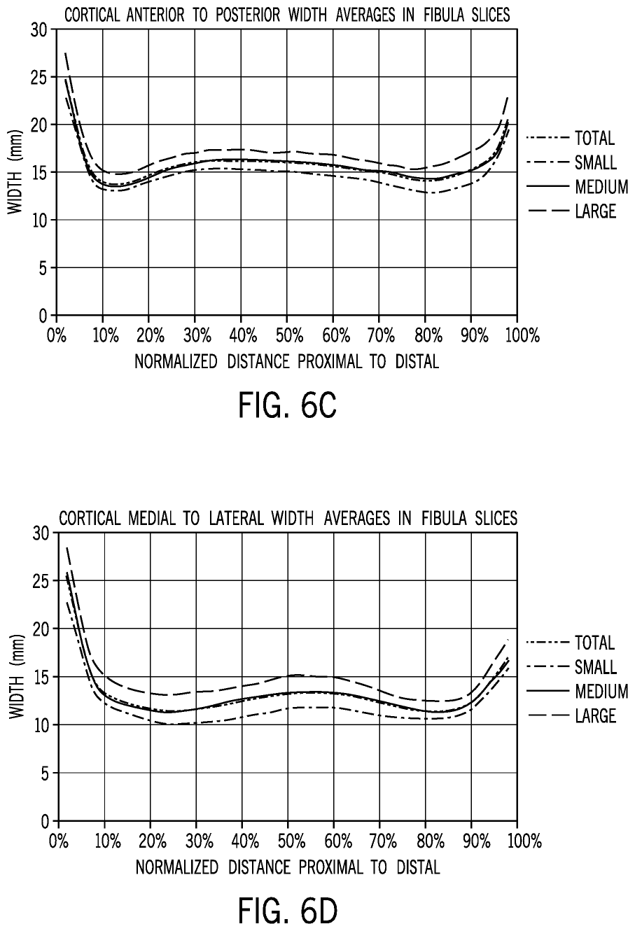

Referring to FIGS. 4A-4D, 5A-5D, and 6A-6D, non-limiting example graphical representations of bone thickness measurements are also shown in FIGS. 4A-4D for tibia, 5A-5D for femur, and 6A-6D for fibula data. Data from any bone may be used to guide implant design for any implant in accordance with the present disclosure.

Implants may take on any form including, but not limited to, joint arthroplasty, trauma, dental implants, and the like. Non-limiting examples include stems for joint arthroplasty, stemless implants for joint arthroplasty, trauma implants including intramedullary nails and plates, sports medicine implants such as anchors and screws, as well as dental implants.

The material properties of the implant, such as a plate, intramedullary nail, and the like, can be tailored to the specific anatomic site. The thickness of the cortices varies significantly based on the specific anatomic location where a plate may be placed. An implant designed with these factors taken into account can result in more physiologic loads to the underlying bone and has the potential to minimize uneven stress distribution and improve fracture healing. Moreover, the material properties of an implant, such as an intramedullary nail may be optimized for patients with different quality bone in specific locations, such as the difference between young patients compared to older patients. This has the potential for wide implementation since the distribution of many fractures constitutes a clear bimodal population. Femur fractures occur most frequently in either high velocity accidents in young males or simple falls in elderly women.

Implant elasticity and the long-term bone integrity associated with adaptive bone remodeling are strongly related. Altering implant material properties can alter elastic properties. Adjusting titanium porosity results in elastic properties closer to those of bone than to solid titanium surfaces. Functionally graded material (FGM), where the mechanical properties can be tailored to vary with position within the material, may be used to adjust the modulus of elasticity within an implant.

In some configurations, the modulus of elasticity of the implant may be modified by adjusting the coating of the implant in regards to type, porosity, thickness, and the like with respect to location on the implant. Modification of elasticity may also include using different coatings in different areas of the implant. Modification of elasticity may also include altering the shape of the implant, such as by matching a fluted, oval, spherical, or other shape to the anatomy of the bone. Modification of elasticity may also include adjusting the modulus of elasticity of the implant material in a zone specific manner.

Surface roughness treatments such as polishing, sandblasting, plasma-spraying, or porous beading may have an impact on the distribution of stresses at the bone-implant interface. In some configurations, the use of surface roughness methods may result in improved stress distribution in peri-implant tissues. In FEA analysis, porous-surfaced implants were shown to distribute stress in a more uniform pattern around the implant compared with smooth surfaced implants. However, these coatings have traditionally been applied in a symmetric manner. Moreover, in these FEA analysis, bone has been simplified and modeled to be symmetric. In accordance with the present disclosure, surface roughness methods may be applied or utilized asymmetrically on an implant. Imaging or modelling the asymmetry in bone architecture may be used to apply asymmetric coatings to an implant to better match the implant to the anatomy and improve stress distribution.

### Non-limiting Example Population Based Design

In some configurations, the method utilizes the bone properties measurements of a wide population of patients. Implants may then be manufactured in mass to fit an average size or configuration rather than being patient specific. In some configurations, different implant size and configuration options may be created based on differing populations. In non-limiting examples, an implant may include characteristics optimized for a younger patient with thicker cortices and good quality bone, and another implant may be optimized for an older patient with bone that has thinner cortices and poorer quality bone. A true centerline measurement generated from the population averages may be used to define the true center of the canal and can be used to design implants that match the shape of the canal and result in more uniform stress distribution.

### Custom Implant Application

Individualized implants may be designed using additive manufacturing and 3D printing to customize the implant for an individual patient in accordance with the present disclosure. The material properties of the implant including coatings, shape, and material properties may be adjusted to create in an implant designed to optimize the bone-implant interface for that individual patient.

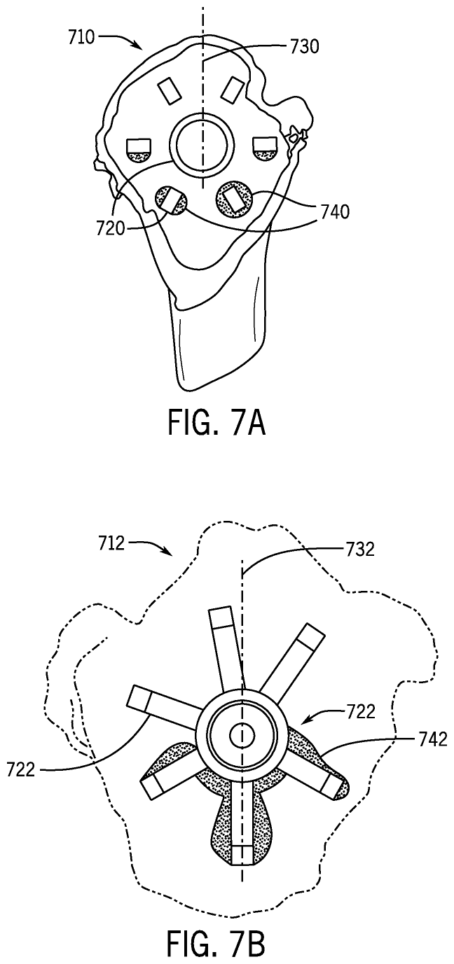

Referring to FIG. 7A, a non-limiting example is shown of a cross section of a bone 710 with a symmetrical implant 720. The symmetrical implant 720 may be symmetrical with respect to a plane of symmetry or reflection plane 730. The symmetrical implant 720 may include an asymmetrical coating 740. Asymmetrical coating 740 includes material properties that are asymmetrical with respect to the reflection plane 730. The asymmetry provides that no reflection plane 730 may be drawn to create bilateral symmetry of the asymmetrical coating 740 on the symmetrical implant 720. The asymmetrical coating 740 may be configured on the symmetrical implant 720 in order to promote bone growth and optimize the bone-implant interface, in accordance with the present disclosure. Asymmetrical coating 740 may be asymmetrical in thickness, location on the implant, porosity, elasticity, or other material parameter, and the like.

Referring to FIG. 7B, a non-limiting example is shown of a cross section of a bone 712 with an asymmetrical implant 722. The asymmetrical implant 722 may be asymmetrical with respect to a plane of symmetry or reflection plane 732. That is, that the asymmetrical implant lacks bilateral symmetry across the reflection plane 732, and that no orientation of the reflection plane 732 results in bilateral symmetry. The asymmetrical implant 722 may include an asymmetrical coating 742. Asymmetrical coating 742 includes material properties that are asymmetrical with respect to the reflection plane 732. The asymmetry provides that no reflection plane 732 may be drawn to create bilateral symmetry of the asymmetrical coating 742 on the asymmetrical implant 722. The asymmetrical coating 742 may be configured on the asymmetrical implant 722 in order to promote bone growth and optimize the bone-implant interface, in accordance with the present disclosure. Asymmetrical coating 742 may be asymmetrical in thickness, location on the implant, porosity, elasticity, or other material parameter, and the like.

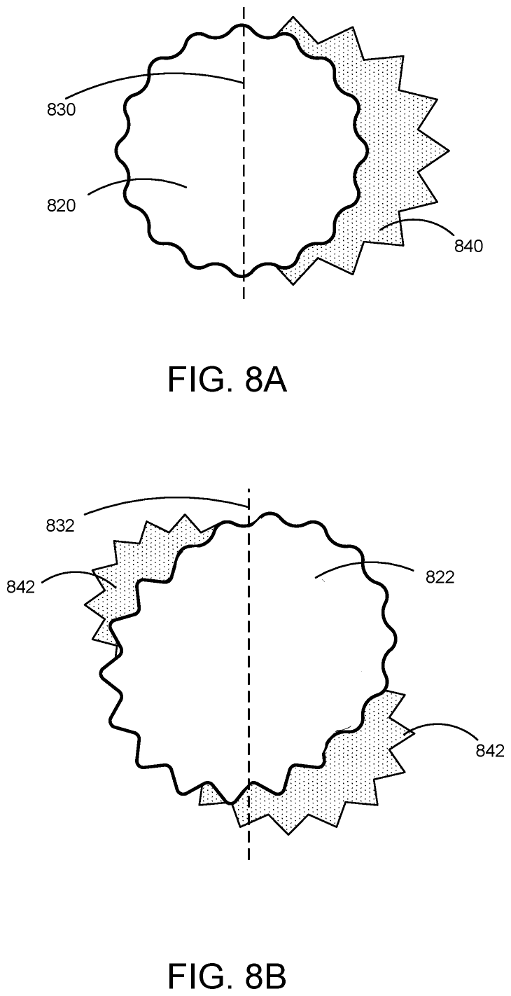

Referring to FIG. 8A, a non-limiting example is shown of a cross section of a symmetrical stem 820 for implantation into a bone. The symmetrical stem 820 may be symmetrical with respect to a plane of symmetry or reflection plane 830. The symmetrical stem 820 may include an asymmetrical coating 840. Asymmetrical coating 840 includes material properties that are asymmetrical with respect to the reflection plane 830. The asymmetry provides that no reflection plane 830 may be drawn to create bilateral symmetry of the asymmetrical coating 840 on the symmetrical stem 820. The asymmetrical coating 840 may be configured on the symmetrical stem 820 in order to promote bone growth and optimize the bone-implant interface, in accordance with the present disclosure. Asymmetrical coating 840 may be asymmetrical in thickness, location on the implant, porosity, elasticity, or other material parameter, and the like.

Referring to FIG. 8B, a non-limiting example is shown of a cross section of an asymmetrical stem 822 for implantation into a bone. The asymmetrical stem 822 may be asymmetrical with respect to a plane of symmetry or reflection plane 832. The asymmetrical stem 822 may include an asymmetrical coating 842. Asymmetrical coating 842 includes material properties that are asymmetrical with respect to the reflection plane 832. The asymmetry provides that no reflection plane 832 may be drawn to create bilateral symmetry of the asymmetrical coating 842 on the asymmetrical stem 822. The asymmetrical coating 842 may be configured on the asymmetrical stem 822 in order to promote bone growth and optimize the bone-implant interface, in accordance with the present disclosure. Asymmetrical coating 842 may be asymmetrical in thickness, location on the implant, porosity, elasticity, or other material parameter, and the like.

### Stress Shielding

One skilled in the art would appreciate that the implant design and methodology may include taking into account the anatomy of a bone of a subject to address stress shielding considerations for the subject. Stress shielding may develop over time in a subject with an implant, and optimizing the bone-implant interface may address stress shielding considerations as well, such as by preventing bone resorption in a subject over time. As described above, implants or components can be asymmetrically designed to better match the associated anatomy as well as to address stress shielding when the bone-implant interface is optimized, such as by quantifying bone density and matching material properties of the implant. Information derived from the methodology can be used to guide the design of the implant resulting in an asymmetric design that optimizes the bone-implant interface. In some configurations for addressing stress shielding, a method for implant design to optimize the bone-implant interface includes an asymmetrical shape of the implant; an asymmetric coating applied to the implant, an asymmetric type of coating applied to the implant; an asymmetric modulus of elasticity of the implant material, and the like.

The present disclosure has described one or more preferred embodiments, and it should be appreciated that many equivalents, alternatives, variations, and modifications, aside from those expressly stated, are possible and within the scope of the invention.

1. A method for manufacturing an orthopedic implant for repairing a part of a bone in a subject, the method comprising:
forming the implant to include at least one material property determined by;
i) obtaining an image of the bone from at least one viewing plane;
ii) orienting on the image a cross section indicating a maximum width of a feature of the bone from a first border of the bone to an opposite second border of the bone;
iii) determining the at least one material property of the implant to optimize the bone-implant interface using the maximum width of the feature of the bone.

2. The method of claim 1, wherein the material property includes at least one of elasticity, surface coating treatment, porosity, thickness, or shape.

3. The method of claim 2, wherein the material property creates an asymmetric implant.

4. The method of claim 2, wherein the at least one material property includes the surface coating treatment of the implant, and wherein the surface coating treatment corresponds to an anatomic location determined by a location of the cross section of the bone.

5. The method of claim 4, wherein the surface coating treatment is asymmetric on the surface of the implant.

6. The method of claim 1, further comprising determining a quality of the bone using the maximum width of the feature of the bone.

7. The method of claim 6, wherein determining the quality of the bone includes determining a score for the quality of the bone in an anatomic location.

8. The method of claim 1, wherein a plurality of cross sections are oriented on the image along a longitudinal axis of the bone.

9. The method of claim 8, wherein bone quality is determined for a plurality of features along the longitudinal axis of the bone using the maximum width of each of the plurality of features.

10. The method of claim 1, wherein the bone is a humerus and a joint that includes the bone is a shoulder.

11. The method of claim 1, further comprising determining the at least one material property of the implant to address stress shielding of the bone.

12. A device for repairing a part of a bone in a subject, the device comprising:
a first section having a first material property;
a second section having a second material property;
the first section being connected to the second section forming an asymmetric implant, wherein the first section and the second section are configured to optimize the bone-implant interface.

13. The device of claim 12, wherein the first material property includes at least one of elasticity, surface coating treatment, porosity, thickness, or shape, and
wherein the second material property includes at least one of elasticity, surface coating treatment, porosity, thickness, or shape.

14. (canceled)

15. The device of claim 12, wherein at least one of the first material property or the second material property includes a surface coating treatment of the device, and wherein the surface coating treatment corresponds to a location of uneven stress distribution, and
wherein the surface coating treatment is asymmetric on the surface of the device, and
wherein the location of uneven stress distribution of the bone corresponds to a location of reduced bone thickness.

16. (canceled)

17. (canceled)

18. The device of claim 12, wherein the bone is a humerus and a joint that includes the bone is a shoulder.

19. The device of claim 12, wherein the first section and the second section are configured to reduce stress shielding.

20. A device for repairing a part of a bone in a subject, the device comprising:
a first section having a first material property;
a second section having a second material property, the first section being connected to the second section; and
wherein at least one of the first material property or the second material property includes a surface coating treatment forming an asymmetric implant configured to optimize the bone-implant interface.

21. (canceled)

22. (canceled)

23. (canceled)

24. (canceled)

25. (canceled)

26. (canceled)

27. The method of claim 1 further comprising:
determining the at least one material property of the implant to reduce stress shielding for the bone using the maximum width of the feature of the bone.

28. (canceled)

29. (canceled)

30. (canceled)

31. (canceled)

32. (canceled)

33. (canceled)

34. (canceled)

35. (canceled)

36. (canceled)

37. The device of claim 20 wherein:
the first section and the second section are configured to reduce stress shielding to a region of the bone.

38. (canceled)

39. (canceled)

40. (canceled)

41. (canceled)

42. (canceled)

43. (canceled)

44. The device of claim 20 wherein:
at least one of the first material property or the second material property includes a surface coating treatment forming an asymmetric implant configured to reduce stress shielding to a region of the bone.

45. (canceled)

46. (canceled)

47. (canceled)

48. (canceled)

49. (canceled)
